(19) United States
(12) Patent Application Publication
(10) Pub. No.: US 2023/0333099 A1
(43) Pub. Date: Oct. 19, 2023

(54) COMPOSITE MAGNETIC NANOMATERIAL BASED ON DNA TETRAHEDRON, PREPARATION THEREFOR AND USE THEREOF

(71) Applicant: NATIONAL INSTITUTE OF METROLOGY,CHINA, Beijing (CN)

(72) Inventors: Rui ZHAI, Beijing (CN); Zhanying CHU, Beijing (CN); Manman ZHU, Beijing (CN); Yang ZHAO, Beijing (CN); Xiaoyun GONG, Beijing (CN); Jie XIE, Beijing (CN); Liqing WU, Beijing (CN); You JIANG, Beijing (CN); Xinhua DAI, Beijing (CN); Xiang FANG, Beijing (CN)

(21) Appl. No.: 18/337,508

(22) Filed: Jun. 20, 2023

Related U.S. Application Data
(63) Continuation-in-part of application No. PCT/CN2022/072553, filed on Jan. 18, 2022.

(30) Foreign Application Priority Data
Jan. 19, 2021 (CN) 202110071495.7

(51) Int. Cl. G01N 33/543; G01N 33/68

(52) U.S. Cl. CPC G01N 33/54346; G01N 33/6848; G01N 33/54326

(57) ABSTRACT

A composite magnetic nanomaterial based on a DNA tetrahedron, preparation therefor and use thereof are provided. The preparation method include the steps of synthesizing a DNA tetrahedron by means of a self-assembly reaction of single-stranded DNA chains, loading magnetic nanoparticles on the surfaces of molybdenum disulfide particles, modifying gold nanoparticles on exposed active sulfur atoms of the molybdenum disulfide particles, modifying the DNA tetrahedron on the gold nanoparticles, and bonding a protein antibody on the DNA tetrahedron. The composite magnetic nanomaterial is used for enriching and detecting low-abundance proteins in serum, and specific low-abundance proteins in the serum can be efficiently and selectively enriched by means of the specific reaction between an antigen and an antibody.

### FIELD

The present disclosure relates to the technical field of functionalized magnetic nanomaterials, in particular to a composite magnetic nanomaterial based on a DNA tetrahedron, preparation therefor and use thereof.

### BACKGROUND

Malignant tumor has become a disease with high morbidity and high mortality that seriously affects human health. In China, the morbidity of the malignant tumor increases by 3.9% every year, and the mortality of the malignant tumor increases by 2.5% every year. Relevant data show that ⅓ of cancers can be radically treated through early detection. However, many cancer patients in China are in the middle and late stage once they are found, and are more difficult to treat. Therefore, development of tumor markers for early cancer diagnosis is of great significance for the diagnosis and treatment of cancer.

Malignant tumor markers in serum usually belong to low-abundance proteins (e.g., HSP90α, a liver cancer marker, is typically present in blood at only about 60 ng/mL), however, proteins in serum are complex and diverse, and the presence of high-abundance proteins will severely interfere with the detection of the low-abundance proteins. Enrichment of the low-abundance proteins in serum by using an antibody is a common method to increase the detection sensitivity of the low-abundance proteins. A solid phase extraction technology can effectively extract a target substance from a complex matrix, and thus has great development potential in the detection of the low-abundance proteins.

A DNA tetrahedron (DNA TET) is a nanomaterial with abundant modification sites and good biocompatibility, and is gradually becoming a research hotspot for DNA nanomaterials. The DNA TET material can be self-assembled by only one step of a thermal denaturation reaction, and the synthesis method is simple with high yield. By using the abundant modification sites in DNA TET, a functional molecule can be bonded to a vertex of the DNA tetrahedron material, wrapped in its cage-like pore structure, or embedded or hung on the edge of a double helix by a self-assembly strategy through the chemical means such as ligand design, or even its structural change can be intelligently controlled by introducing a hairpin loop structure, etc. The DNA tetrahedron nanomaterial can effectively control the orientation and spacing of a modified group or molecule, and can achieve the specific capture of low-abundance target substances, and is especially suitable for specific interaction with low-abundance substances in the complex matrix.

### SUMMARY

The present disclosure aims to overcome the shortcomings in the prior art, and provide a composite magnetic nanomaterial based on a DNA tetrahedron, preparation therefor and use thereof. The composite magnetic nanomaterial can efficiently and selectively enrich specific low-abundance proteins in serum by means of a specific reaction between an antigen and an antibody, and the material takes a magnetic nanomaterial as a matrix, and thus has the characteristic of being simple, convenient and fast to use, and the treatment time of a complex matrix in serum is greatly shortened.

The present disclosure adopts the following technical solutions:
provided is a composite magnetic nanomaterial based on a DNA tetrahedron, wherein the composite magnetic nanomaterial comprises molybdenum disulfide particles, magnetic nanoparticles coating the surfaces of the molybdenum disulfide particles, gold nanoparticles modified on exposed active sulfur atoms on the molybdenum disulfide particles by a reaction of a Au—S bond, a sulfhydryl-containing DNA tetrahedron stably immobilized on the gold nanoparticles, and a protein antibody bonded to the DNA tetrahedron by a reaction of a functional group on a vertex of the DNA tetrahedron.

Further, the magnetic nanoparticles are ferroferric oxide magnetic nanoparticles with a particle size of 20-800 nm, such as 40 nm. The particle size of the gold nanoparticles has no special requirement.

Further, the molybdenum disulfide particles have a spherical structure with a particle size of 1-50 μm, preferably 1-20 μm, more preferably 5-10 μm.

Further, the molybdenum disulfide particles have a lamellar structure inside, and the lamellar thickness is 0.1-2 nm, preferably 0.2-1 nm.

Further, four DNA single strands of the DNA tetrahedron are synthesized by self-assembly, and each DNA single strand comprises 16-160 deoxyribonucleotide monomers.

Further, the DNA tetrahedron is formed by four DNA single strands each having a concentration of 1 μmol/L through base complementary pairing.

Further, a 3′ end or 5′ end of each DNA single strand has a functional group, and the functional group may be sulfhydryl, carboxyl, an aldehyde group, an epoxy group or amino; wherein sulfhydryl is used for a reaction of the DNA tetrahedron with the gold nanoparticles in the magnetic nanomaterial, and carboxyl, the aldehyde group, the epoxy group or amino is used for bonding between the DNA tetrahedron and an antibody.

Further, the protein antibody may be a monoclonal antibody or polyclonal antibody of low-abundance proteins in serum.

A preparation method for a composite magnetic nanomaterial based on a DNA tetrahedron comprises the following steps of:
S1, loading ferroferric oxide magnetic nanoparticles on the surfaces of molybdenum disulfide particles to obtain a product I: MoS₂@Fe₃O₄;
S2, modifying gold nanoparticles on exposed active sulfur atoms of the product I by the interaction of a “Au—S” bond to obtain a product II: MoS₂@Fe₃O₄@AuNPs;
S3, modifying a DNA tetrahedron of which three vertices contain sulfhydryl on the gold nanoparticles in the product II by the interaction of “Au—S” bonds to obtain a product III: MoS₂@Fe₃O₄@AuNPs@DNA TET; and
S4, activating carboxyl modified on the DNA tetrahedron in the product III, and incubating the activated product III with a protein antibody solution to connect the protein antibody to the composite magnetic nanomaterial.

Further, in the step S1, the molybdenum disulfide may be prepared according to the following conventional method: dissolving Na₂MoO₄·2H₂O, (NH₂)₂CS and PEG-20,000 in deionized water, adding the resulting solution to a stainless steel reactor, and carrying out a reaction at a high temperature.

Further, the magnetic nanoparticles may be Fe₃O₄ magnetic nanoparticles, and the Fe₃O₄ magnetic nanoparticles may be prepared according to a conventional method such as adding anhydrous sodium acetate to a solution of ferric chloride hexahydrate in ethylene glycol to obtain a mixed solution; and heating the mixed solution, and performing cooling and drying to obtain the Fe₃O₄ magnetic nanoparticles. The heating may be performed at a temperature of 220° C. for 8-12 h, in particular 8 h.

Further, in the step S1, the magnetic nanoparticles are loaded on the surface of the molybdenum disulfide by the following steps of: placing a MoS₂ nanomaterial, FeCl₃·6H₂O and trisodium citrate in a centrifuge tube, and adding ethylene glycol to the centrifuge tube; and after ultrasonic dispersion, adding sodium acetate, then adding dropwise aqueous ammonia while stirring, transferring the mixed solution after the reaction to a stainless steel reactor, and carrying out a reaction at a high temperature to obtain the product I.

Further, in the step S2, the gold nanoparticles can be modified on the surface of the product I by the following steps of: adding deionized water to the MoS₂@Fe₃O₄ composite, and adding a solution of HAuCl₄ and a solution of sodium citrate; quickly adding a freshly prepared NaBH₄ solution thereto with vigorous stirring; and after another 30-minute mechanical stirring, allowing the mixed solution to stand in the dark for 16 h to obtain the product II.

Further, in the step S3, the DNA tetrahedron is prepared by self-assembly through base complementary pairing of four DNA single strands each having a concentration of 1 μmol/L; sulfhydryl of the DNA tetrahedron is activated by adding DTT; and the activated DNA tetrahedron is added to the product II prepared in the step S2, and the product III is obtained after reaction.

Further, a ratio of a molar concentration of DTT added to a molar concentration of DNA may be (10-100):1, and in particular, may preferably be 50:1. The reaction may be carried out for 16 h.

Further, in the step S4, EDC and NHS in a certain ratio are added to the activated product III and the protein antibody solution to activate carboxyl modified on the DNA tetrahedron, wherein the ratio of EDC to NHS is 1:(1-5), and in particular, may preferably be 1:2. The incubation reaction may be carried out at 37° C. for 1 h. Wherein EDC is 1-(3-dimethylaminopropyl)-3-ethylcarbodiimide hydrochloride, and NHS is N-hydroxysuccinimide.

Provided is use of a composite magnetic nanomaterial based on a DNA tetrahedron, wherein the composite magnetic nanomaterial is used for protein-specific enrichment and detection.

Further, the enrichment and detection comprises the steps of mixing the synthesized composite magnetic nanomaterial with a sample containing a target protein, incubating the mixture for a certain period of time, performing magnetic separation, removing a supernatant, subjecting the composite magnetic nanomaterial enriched with the target protein to enzymatic digestion, and performing mass spectrometry detection.

The beneficial effects of the present disclosure are as follows: the DNA tetrahedron that has good biocompatibility and is likely to be stably immobilized on the surface of a nanomaterial is loaded on the surface of the nanomaterial by a simple two-step reaction of the “Au—S” bond, and the material is easy of synthesis and environmentally friendly; and highly efficient and highly selective enrichment of low-abundance proteins in a complex matrix can be realized by the protein antibody loaded onto the composite magnetic nanomaterial.

### BRIEF DESCRIPTION OF THE DRAWINGS

FIG. 1 shows a schematic diagram of a synthetic route for a magnetic nanomaterial based on

FIG. 2 shows SEM photographs and TEM photographs of products MoS₂ and MoS₂@Fe₃O₄ in a synthesis process of Example 1, wherein panel A is a SEM photograph of MoS₂, panel B is a SEM photograph of MoS₂@Fe₃O₄, panel C is a TEM photograph of MoS₂, and panel D is a TEM photograph of MoS₂@Fe₃O₄.

FIG. 3 shows four single-stranded DNA sequences of the synthesized DNA tetrahedron in Example 1.

FIG. 4 shows a magnetic characterization diagram of a product MoS₂@Fe₃O₄@AuNPs in the synthesis process of Example 1.

FIG. 5 shows UV absorption spectra of products MoS₂@Fe₃O₄@AuNPs and MoS₂@Fe₃O₄@AuNPs@DNA TET in the synthesis process of Example 1.

FIG. 6 shows MALDI-TOF MS (Matrix-Assisted Laser Desorption/Ionization Time of Flight Mass Spectrometry) examining the material sensitivity, wherein a HSP90α solution has a concentration of 10 ng/mL.

### DETAILED DESCRIPTION OF THE EMBODIMENTS

Hereinafter, specific examples of the present disclosure will be described in detail with reference to specific drawings. It should be noted that the technical features described in the following examples or a combination of the technical features should not be considered as isolated, and they may be combined with each other to achieve better technical effects.

The experimental methods used in the following examples are conventional methods unless otherwise specified.

Materials, reagents and the like used in the following examples are commercially available unless otherwise specified.

A composite magnetic nanomaterial based on a DNA tetrahedron according to an example of the present disclosure comprises molybdenum disulfide particles, magnetic nanoparticles coating the surfaces of the molybdenum disulfide particles, gold nanoparticles modified on exposed active sulfur atoms on the molybdenum disulfide particles by a reaction of a Au—S bond, a DNA tetrahedron of which three vertices contain sulfhydryl which is stably immobilized on the gold nanoparticles, and a protein antibody connected to the DNA tetrahedron by a reaction of carboxyl at one remaining vertex of the DNA tetrahedron.

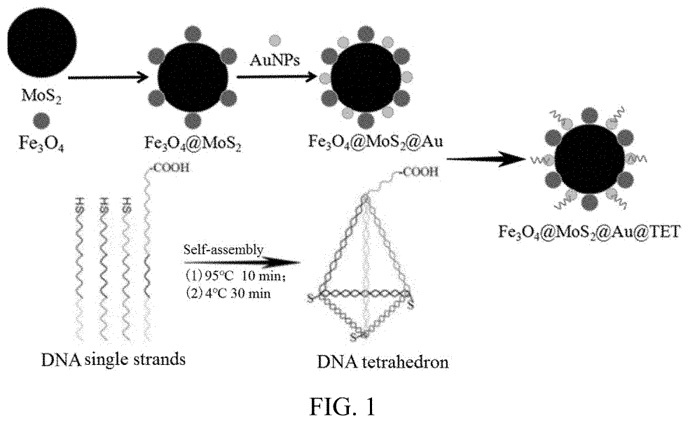

As shown in FIG. 1, a preparation method for a composite magnetic nanomaterial based on a DNA tetrahedron according to an example of the present disclosure comprises the following steps of:
S1, loading ferroferric oxide magnetic nanoparticles on the surfaces of molybdenum disulfide particles to obtain a product I: MoS₂@Fe₃O₄;
S2, modifying gold nanoparticles on exposed active sulfur atoms of the product I by the interaction of a “Au—S” bond to obtain a product II: MoS₂@Fe₃O₄@AuNPs;
S3, modifying a DNA tetrahedron of which three vertices contain sulfhydryl on the gold nanoparticles in the product II by the interaction of “Au—S” bonds to obtain a product III: MoS₂@Fe₃O₄@AuNPs@ DNA TET; and
S4, activating carboxyl modified on the DNA tetrahedron in the product III, and incubating the activated product III with a protein antibody solution to connect the protein antibody to the composite magnetic nanomaterial.

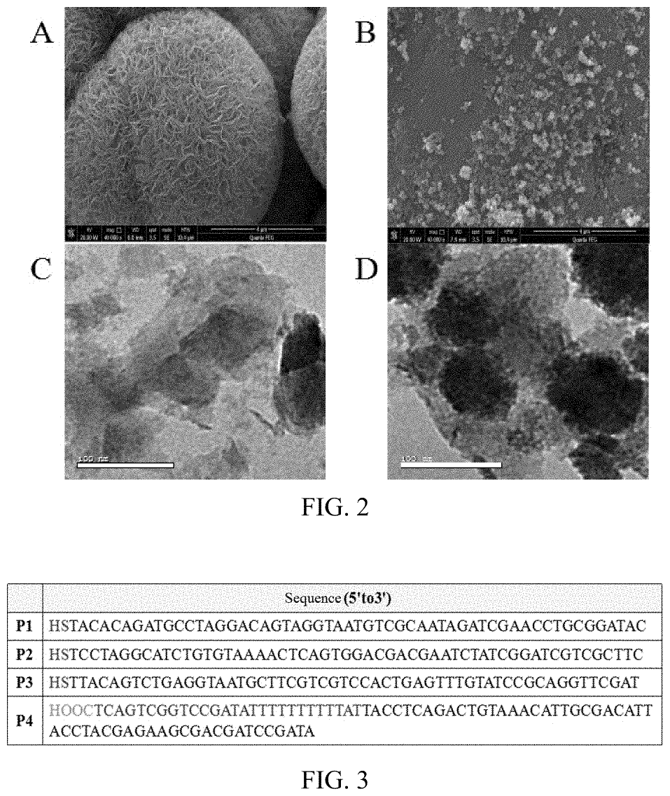

The molybdenum disulfide (MoS₂) used in the following examples is prepared by the following steps of: dissolving 1.210 g of Na₂MoO₄·2H₂O, 1.520 g of (NH₂)₂CS and 0.030 g of PEG-20,000 in 30 mL of deionized water; performing stirring for 30 min, and performing ultrasonic treatment for 30 min until a uniform transparent solution is obtained; transferring the solution to a 50 mL stainless steel reactor, heating the solution to 220° C. in an air-blast drying oven, and carrying out a reaction for 24 h. After the reaction is completed, cooling the resulting reaction solution to room temperature, and performing centrifugation at 1500 r/min for 15 min to separate out a precipitate; then sequentially washing with 30 mL of deionized water twice, washing with 30 mL of anhydrous ethanol twice, and washing with 30 mL of deionized water for three times, performing centrifugation, taking the obtained precipitate to be dried in a vacuum oven at 60° C. for 6 h, and storing the dried precipitate for later use. As shown in FIG. 2, molybdenum disulfide has a spherical structure, a smooth surface, a particle size of 7.5-8 μm, and a lamellar structure inside, and the lamellar thickness is 0.02 μm.

The product I: MoS₂@Fe₃O₄ used in the following examples is prepared by the following steps of: weighing 30 mg of MoS₂, 100 mg of FeCl₃·6H₂O and 30 mg of trisodium citrate to be placed in a 50 mL centrifuge tube, and adding 30 mL of ethylene glycol to the centrifuge tube; performing ultrasonic dispersion for 2 h, adding 700 mg of sodium acetate to the centrifuge tube, and performing mechanical stirring for 30 min so that sodium acetate is fully dissolved; adding dropwise 300 μl of aqueous ammonia while stirring, and then continuing to perform mechanical stirring for 10 min; transferring the mixed solution after the reaction to a 50 mL stainless steel reactor, heating the solution to 220° C. in an air-blast drying oven, and carrying out a reaction for 9 h; after the reaction is completed, cooling the resulting reaction solution to room temperature, and performing separation by using a magnet to obtain a product, i.e., MoS₂@Fe₃O₄; sequentially washing the obtained precipitate with anhydrous ethanol and deionized water twice respectively, and performing separation by using a magnet after each washing; and drying the well washed precipitate in a vacuum oven at 60° C. for 10 h. As shown in FIG. 2, after magnetic nanoparticles are immobilized, it can be observed that a large number of microspheres having a diameter of about 0.08 μm are distributed on the surface of a ultra-thin two-dimensional molybdenum disulfide nanomaterial.

The product II: MoS₂@Fe₃O₄@AuNPs used in the following examples are prepared by the following steps of: preparing a 0.01 mol/L solution of HAuCl₄ and a 0.01 mol/L solution of sodium citrate; weighing 19 mg of NaBH₄, and adding 5 mL of deionized water (ice water) theretoto prepare a 0.1 mol/L NaBH₄ solution (being used right after being ready); weighing 65 mg of the MoS₂@Fe₃O₄ composite to be placed in a round bottom flask, adding 40 mL of deionized water to the round bottom flask, and performing mechanical stirring to uniformly suspend the material in the deionized water; adding 2 mL of the 0.01 mol/L solution of HAuCl₄ and 2 mL of the 0.01 mol/L solution of sodium citrate thereto while stirring; continuing stirring for 10 min, and quickly adding 2 mL of the freshly prepared 0.1 mol/L NaBH₄ solution thereto with vigorous stirring; continuing to perform mechanical stirring for 30 min, and allowing the mixed solution to stand in the dark for 16 h; separating the sample after standing by using a magnet to obtain a product, i.e., MoS₂@Fe₃O₄@AuNPs; and sequentially washing the obtained precipitate with anhydrous ethanol, and deionized water twice respectively, and performing separation by using a magnet after each washing.

The DNA tetrahedron used in the following examples is prepared by the following steps of: designing four DNA single strands as shown in FIG. 3, each single-stranded DNA being prepared to a concentration of 100 μmol/L; adding 1 μL of the solution for each single strand to 96 μL of a TE buffer to prepare a final concentration of 1 μmol/L for each single strand; and performing self-assembly to form the DNA tetrahedron by maintaining for 10 min at 95° C., maintaining for 30 min at 4° C. It should be noted that carboxyl at a 5′ end of a DNA sequence illustrated by P4 in FIG. 3 is a preferred functional group in this example, and this functional group can be replaced by an epoxy group, an aldehyde group, amino, or the like according to actual needs. Appropriate modifications based on the single strands of DNA designed in this example should also fall within the scope of protection of this patent application.

The product III: MoS₂@Fe₃O₄@AuNPs@DNA TET used in the following examples is prepared by the following steps of: weighing 18 mg of the MoS₂@Fe₃O₄@AuNPs composite, adding 100 μl of the above prepared DNA tetrahedron, 200 μl of a TE buffer, and 10 μl of a 50 mmol/L NaCl solution thereto, carrying out a reaction, and incrementally adding 5 μl of the 50 mmol/L NaCl solution thereto every other 1 h for 4 times; and after the reaction in a environment of 4° C. for 12 h, storing a sample in a refrigerator of 4° C. for later use. As shown in FIG. 5, MoS₂@Fe₃O₄@AuNPs have no significant absorption peak in a wavelength range of 230-280 nm, and the MoS₂@Fe₃O₄@AuNPs@DNA TET composite exhibits a stronger absorption peak at 259 nm compared with the MoS₂@Fe₃O₄@AuNPs material, which is consistent with the UV absorption of DNA at 260 nm, indicating that DNA tetrahedron has been successfully loaded onto the composite MoS₂@Fe₃O₄@AuNPs.

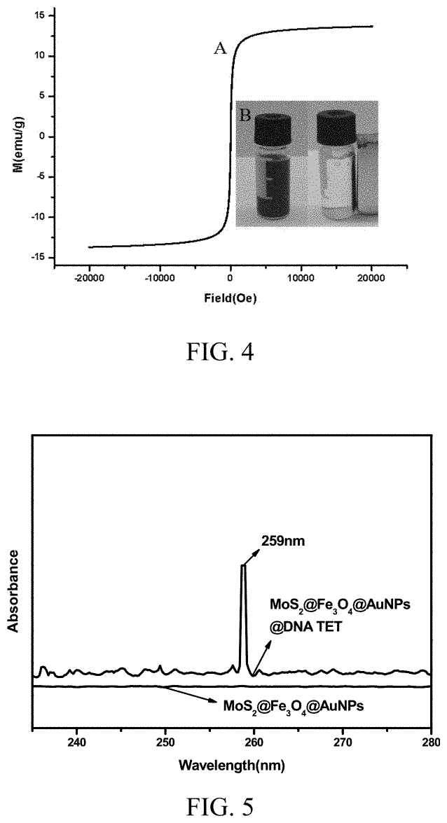

The MoS₂@Fe₃O₄@AuNPs@DNA TET@Ab used in the following examples is prepared by the following steps of: preparing a 0.1 mol/L MES buffer solution (pH=6) to dissolve EDC and NHS, pipetting 1 mg of a material, adding a solution of EDC and NHS in a molar ratio of 2:1 to the material, activating the material for 30 min, performing sucking to remove a supernatant, and washing the material for 3 times with a TE buffer; pipetting 100 μl of an antibody solution, adding the antibody solution to the material, and then adding 300 μl of a TE buffer; and incubating the material in a refrigerator of 4° C. for 12 h. As shown in FIG. 4, a hysteresis curve of the resulting product shows that the material has good paramagnetic capacity, and can be separated quickly by using a magnet. It should be noted that a carboxyl activation solution enumerated in this step is a preferred solution for this example, and in practice, if other functional groups are used at the 5′ end of a P4 strand in the single strands of DNA, this experimental step should also be adjusted accordingly. For example, when the carboxyl is replaced with an epoxy group or an aldehyde group, a reaction step of activation with EDC and NHS does not need to be performed.

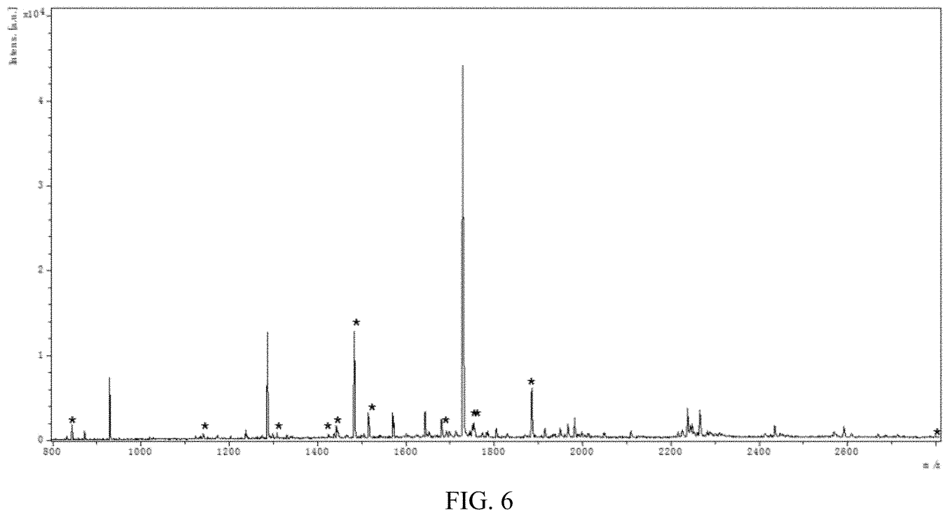

Taking HSP90α protein as an example, the performance of the magnetic composite nanomaterial for enriching low-abundance proteins in a complex matrix in an actual sample is examined. The synthesized material is applied to the enrichment of HSP90α in plasma of a cancer patient. 100 μl of the plasma of the cancer patient is pipetted into 900 μl of a PBS buffer. 1 ml of the solution is added to 1 mg of the magnetic composite for a specific enrichment reaction. A supernatant after the reaction is removed by sucking, the material is washed with a washing solution, an enzymolysis reaction is performed, and 2 μl of the enzymatic hydrolysate after the reaction is pipetted for MALDI-TOF detection. FIG. 6 is MALDI-TOF MS of HSP90α-enriched plasma of the cancer patient after enzymolysis. Ten specific peptides of HSP90α can be detected. This result indicates that the prepared magnetic composite has the function of specifically enriching HSP90α in an actual serum sample, providing a better method for separation and enrichment prior to subsequent mass spectrometry detection.

The magnetic composite nanomaterial prepared in the present disclosure consists of the molybdenum disulfide particles, the magnetic nanoparticles coating the surfaces of the molybdenum disulfide particles, and the gold nanoparticles modified on the exposed active sulfur atoms on the molybdenum disulfide material by the two-step reaction of the “Au—S” bond, the DNA tetrahedron of which three vertices contain sulfhydryl being stably immobilized on the surfaces of the gold nanoparticles. The protein antibody is loaded onto the material by reactive connection of carboxyl on one remaining vertex of the DNA tetrahedron to amino on the antibody. The synthesized material according to the present disclosure is simple and clean in method; and highly efficient and highly selective enrichment of low-abundance proteins in a complex matrix can be realized by the protein antibody loaded onto the magnetic composite nanomaterial.

Although several examples of the present disclosure have been given herein, those skilled in the art should understand that changes may be made to the examples herein without departing from the spirit of the present disclosure. The above examples are illustrative only, and should not be construed as a limitation of the scope of the present disclosure.

1. A composite magnetic nanomaterial based on a DNA tetrahedron, wherein comprising molybdenum disulfide particles, magnetic nanoparticles coating the surfaces of the molybdenum disulfide particles, gold nanoparticles modified on exposed active sulfur atoms on the molybdenum disulfide particles by a reaction of a Au—S bond, a sulfhydryl-containing DNA tetrahedron stably immobilized on the gold nanoparticles, and a protein antibody bonded to the DNA tetrahedron by a reaction of a functional group on a vertex of the DNA tetrahedron.

2. The composite magnetic nanomaterial based on the DNA tetrahedron according to claim 1, wherein the magnetic nanoparticles are ferroferric oxide magnetic nanoparticles with a particle size of 20-800 nm.

3. The composite magnetic nanomaterial based on the DNA tetrahedron according to claim 2, wherein the molybdenum disulfide particles have a spherical structure with a particle size of 1-20 m, and the molybdenum disulfide particles have a lamellar structure inside, and the lamellar thickness is 0.1-2 nm.

4. The composite magnetic nanomaterial based on the DNA tetrahedron according to claim 1, wherein four DNA single strands of the DNA tetrahedron are synthesized by self-assembly, and each DNA single strand comprises 16-160 deoxyribonucleotide monomers.

5. The composite magnetic nanomaterial based on the DNA tetrahedron according to claim 4, wherein a 3′ end or 5′ end of each DNA single strand has the functional group, and the functional group is sulfhydryl, carboxyl, an aldehyde group, an epoxy group or amino; wherein sulfhydryl is used for a reaction of the DNA tetrahedron with the gold nanoparticles in the composite magnetic nanomaterial, and carboxyl, the aldehyde group, the epoxy group or amino is used for bonding between the DNA tetrahedron and the protein antibody.

6. The composite magnetic nanomaterial based on the DNA tetrahedron according to claim 1, wherein the protein antibody is a monoclonal antibody or polyclonal antibody of low-abundance proteins in serum.

7. A preparation method for a composite magnetic nanomaterial based on a DNA tetrahedron, wherein comprising the following steps of:
S1, loading ferroferric oxide magnetic nanoparticles on the surfaces of molybdenum disulfide particles to obtain a product I: MoS2@Fe3O4;
S2, modifying gold nanoparticles on exposed active sulfur atoms of the product I by the interaction of a “Au—S” bond to obtain a product II: MoS2@Fe3O4@AuNPs;
S3, modifying a DNA tetrahedron of which vertices contain sulfhydryl on the gold nanoparticles in the product II by the interaction of “Au—S” bonds to obtain a product III: MoS2@Fe3O4@AuNPs@DNA TET; and
S4, activating carboxyl modified on the DNA tetrahedron in the product III, and incubating the activated product III with a protein antibody solution to connect the protein antibody to the composite magnetic nanomaterial.

8. The preparation method for the composite magnetic nanomaterial based on the DNA tetrahedron according to claim 7, wherein in the step S3, the DNA tetrahedron is prepared by self-assembly of four DNA single strands through base complementary pairing; sulfhydryl of the DNA tetrahedron is activated by adding DTT; and the activated DNA tetrahedron is added to the product II prepared in the step S2, and the product III is obtained after reaction.

9. The preparation method for the composite magnetic nanomaterial based on the DNA tetrahedron according to claim 7, wherein in the step S4, EDC and NHS in a certain ratio are added to the product III to activate carboxyl modified on the DNA tetrahedron, wherein the ratio of EDC to NHS is 1:(1-5); wherein EDC is 1-(3-dimethylaminopropyl)-3-ethylcarbodiimide hydrochloride, and NHS is N-hydroxysuccinimide.

10. A method for protein-specific detection, comprising: mixing the composite magnetic nanomaterial prepared according to claim 1 with a sample containing a target protein, incubating the mixture for a certain period of time, performing magnetic separation, removing a supernatant, subjecting the composite magnetic nanomaterial enriched with the target protein to enzymatic digestion, and performing mass spectrometry detection.
